(19) United States
(12) Patent Application Publication
(10) Pub. No.: US 2022/0073864 A1
(43) Pub. Date: Mar. 10, 2022

(54) FIBER SHEET, METHOD FOR MANUFACTURING FIBER SHEET, AND CELL CULTURE CHIP

(71) Applicant: Panasonic Intellectual Property Management Co.,Ltd., Osaka (JP)

(72) Inventors: NORIHITO TSUKAHARA, Kyoto (JP); TAICHI NAKAMURA, Osaka (JP); KOUJI IKEDA, Hyogo (JP); KIYOTAKA TSUJI, Osaka (JP)

(21) Appl. No.: 17/392,423

(22) Filed: Aug. 3, 2021

(30) Foreign Application Priority Data
Sep. 7, 2020 (JP) 2020-149849

(51) Int. Cl. C12N 1/04; B32B 5/12; B32B 7/10; B32B 5/26

(52) U.S. Cl. CPC C12N 1/04; B32B 5/12; B32B 7/10; B32B 5/266; C12N 2513/00; B32B 2250/03; C12N 2533/54; C12N 2533/78; B32B 2262/02; B32B 2250/20; C12N 2533/30

(57) ABSTRACT

A fiber sheet of the present disclosure includes: a first fiber layer including a plurality of first fibers, the plurality of first fibers comprising a thermoplastic polymer and arranged side by side in a first direction; a second fiber layer including a plurality of second fibers, the plurality of second fibers comprising a thermoplastic polymer and arranged side by side in a second direction intersecting the first direction, and disposed to face the first fiber layer; and a nanofiber layer including nanofibers, the nanofibers comprising any one of a thermoplastic polymer, a thermosetting polymer, a biodegradable polymer, and a biological polymer, the nanofiber layer disposed to be in contact with the first fiber layer and the second fiber layer, in which the nanofiber layer is heat-welded to the first fiber layer and the second fiber layer.

### BACKGROUND

### 1. Technical Field

The present disclosure relates to a fiber sheet, a method for manufacturing a fiber sheet, and a cell culture chip.

### 2. Description of the Related Art

In recent years, nanofiber sheets made of ultrafine fibers (nanofibers) having a fiber diameter of about 1 nm to 100 nm have been used as scaffolding materials or filters for filtration in cell culture.

For example, Japanese Patent No. 6452249 discloses a culture base material formed by applying nanofibers made of a biological polymer to gauze.

### SUMMARY

A fiber sheet according to one aspect of the present disclosure includes:

a first fiber layer including a plurality of first fibers, the plurality of first fibers comprising a thermoplastic polymer and arranged side by side in a first direction;

a second fiber layer including a plurality of second fibers, the plurality of second fibers comprising a thermoplastic polymer and arranged side by side in a second direction intersecting the first direction, and disposed to face the first fiber layer; and

a nanofiber layer including nanofibers, the nanofibers comprising any one of a thermoplastic polymer, a thermosetting polymer, a biodegradable polymer, and a biological polymer, the nanofiber layer being disposed to be in contact with the first fiber layer and the second fiber layer,

in which the nanofiber layer is heat-welded to the first fiber layer and the second fiber layer.

A method for manufacturing a fiber sheet according to another aspect of the present disclosure includes:

arranging a plurality of first fibers, which comprise a thermoplastic polymer side by side in a first direction to form a first fiber layer on a surface of a film base material;

forming a nanofiber layer, which contains nanofibers, which comprise any one of a thermoplastic polymer, a thermosetting polymer, a biodegradable polymer, and a biological polymer, on the first fiber layer;

arranging a plurality of second fibers, which comprise a thermoplastic polymer side by side in a second direction intersecting the first direction and arranging the plurality of second fibers to face the first fiber layer to form a second fiber layer on the nanofiber layer;

heating the film base material on which the first fiber layer, the nanofiber layer, and the second fiber layer are formed to heat-weld each of portions at which the nanofibers and the plurality of first fibers are in contact with each other and portions at which the nanofibers and the plurality of second fibers are in contact with each other; and

peeling off the film base material from a structure including the first fiber layer, the nanofiber layer, and the second fiber layer, which are heat-welded.

A method for manufacturing a fiber sheet according to still another aspect of the present disclosure includes:

arranging a plurality of first fibers, which comprise a thermoplastic polymer, side by side in a first direction to form a first fiber layer on a surface of a film base material;

arranging a plurality of second fibers, which comprise a thermoplastic polymer, side by side in a second direction intersecting the first direction and arranging the plurality of second fibers to face the first fiber layer to form a second fiber layer on the first fiber layer;

heating the film base material on which the first fiber layer and the second fiber layer are formed to heat-weld portions at which the plurality of first fibers and the plurality of second fibers intersect and are in contact with each other;

forming a nanofiber layer, which includes nanofibers comprising any one of a thermoplastic polymer, a thermosetting polymer, a biodegradable polymer, and a biological polymer on the first fiber layer and the second fiber layer which are formed on the film base material and heat-welded;

heating the film base material on which the first fiber layer, the second fiber layer, and the nanofiber layer are formed to heat-weld each of portions at which the nanofibers and the plurality of first fibers are in contact with each other and portions at which the nanofibers and the plurality of second fibers are in contact with each other; and

peeling off the film base material from a structure including the first fiber layer, the second fiber layer, and the nanofiber layer, which are heat-welded.

### BRIEF DESCRIPTION OF THE DRAWINGS

FIG. 1 is a schematic view showing an example of a fiber sheet according to a first exemplary embodiment;

FIG. 2 is a cross-sectional view taken along a line A-A of the fiber sheet of FIG. 1;

FIG. 3 is a flowchart showing a method for manufacturing the fiber sheet of FIG. 1;

FIG. 4A is a view showing an example of a manufacturing process of the method for manufacturing the fiber sheet of FIG. 3;

FIG. 4B is a view showing an example of a manufacturing process of the method for manufacturing the fiber sheet of FIG. 3;

FIG. 4C is a view showing an example of a manufacturing process of the method for manufacturing the fiber sheet of FIG. 3;

FIG. 4D is a view showing an example of a manufacturing process of the method for manufacturing the fiber sheet of FIG. 3;

FIG. 4E is a view showing an example of a manufacturing process of the method for manufacturing the fiber sheet of FIG. 3;

FIG. 4F is a view showing an example of a manufacturing process of the method for manufacturing the fiber sheet of FIG. 3;

FIG. 4G is a view showing an example of a manufacturing process of the method for manufacturing the fiber sheet of FIG. 3;

FIG. 5 is a schematic cross-sectional view of a fiber sheet according to a modification example of the first exemplary embodiment;

FIG. 6 is a schematic view showing an example of a fiber sheet according to a second exemplary embodiment;

FIG. 7 is a cross-sectional view taken along a line B-B of the fiber sheet of FIG. 6;

FIG. 8 is a flowchart showing a method for manufacturing the fiber sheet of FIG. 6;

FIG. 9A is a view showing an example of a manufacturing process of the method for manufacturing the fiber sheet of FIG. 8;

FIG. 9B is a view showing an example of a manufacturing process of the method for manufacturing the fiber sheet of FIG. 8;

FIG. 9C is a view showing an example of a manufacturing process of the method for manufacturing the fiber sheet of FIG. 8;

FIG. 9D is a view showing an example of a manufacturing process of the method for manufacturing the fiber sheet of FIG. 8;

FIG. 9E is a view showing an example of a manufacturing process of the method for manufacturing the fiber sheet of FIG. 8;

FIG. 9F is a view showing an example of a manufacturing process of the method for manufacturing the fiber sheet of FIG. 8;

FIG. 9G is a view showing an example of a manufacturing process of the method for manufacturing the fiber sheet of FIG. 8;

FIG. 9H is a view showing an example of a manufacturing process of the method for manufacturing the fiber sheet of FIG. 8;

FIG. 10 is a schematic decomposition view showing an example of a cell culture chip according to a third exemplary embodiment; and

FIG. 11 is a cross-sectional view of the cell culture chip of FIG. 10.

### DETAILED DESCRIPTIONS

### Background to the Present Disclosure

In recent years, nanofiber sheets made of ultrafine fibers (nanofibers) having a fiber diameter of about 1 nm to 100 nm have been used as scaffolding materials or filters for filtration in cell culture.

In the field of cell culture, particularly 3D cell culture, which mimics the growth morphology of biological cells in vitro, such as construction of biological organs while growing in cells in three dimensions (3D), is in the limelight. As a scaffolding material for carrying out the 3D cell culture, attention is increasing to nanofibers that can supply oxygen and nutrients required for target cells and maintain a stable shape.

As a scaffolding material that enables the 3D cell culture, for example, a base material disclosed in Japanese Patent No. 6452249 which is formed by applying nanofibers to a support such as gauze is known. Cells are cultured on this base material.

Nanofibers generally have weak physical strength, and when they are used as a scaffolding material, it is difficult to handle them, and there are problems in terms of handling. In Japanese Patent No. 6452249, gauze or the like is used as a support for nanofibers to increase the physical strength of a base material and improve usability.

However, in the structure of the base material disclosed in Japanese Patent No. 6452249, nanofibers made of a biological polymer are only attached to a surface layer such as gauze that is a support. Therefore, the nanofibers and the support such as gauze are not physically and chemically bonded to each other, and the nanofibers are easily peeled off from the support during handling. Furthermore, there is a problem in terms of quality that the peeled off nanofibers become foreign substances during cell culture, and stable culture cannot be performed. The culture base material disclosed in Japanese Patent No. 6452249 still has room for improvement in terms of quality.

Furthermore, in the base material disclosed in Japanese Patent No. 6452249, the gauze or the like constituting the support is a structure irregular with respect to a plane direction and a thickness direction of the support. Therefore, the gauze or the like becomes a cause that hinders the spread of seeded cells in the plane direction, and there is a problem that it is difficult to obtain a uniform cell membrane in the plane direction.

Furthermore, for example, when two types of cells, intestinal cells and vascular endothelial cells, are co-cultured above and below a scaffolding material, it is desirable that upper and lower cells be separated and in contact with each other to more accurately imitate the organ function in the living body. The thickness of the scaffolding material becomes a cause that hinders the contact between the upper and lower cells, and thus is required to be as small as possible. However, in the base material disclosed in Japanese Patent No. 6452249, the thickness of the gauze itself, which is the support, is 100 μm or greater. Accordingly, there is a problem that it is difficult to use the gauze as a thin scaffolding material having a size of 50 μm or less, which is suitable for co-culture of cells.

Therefore, the inventors of the present disclosure have studied to provide a fiber sheet that can be used as a scaffolding material in cell culture, a high-performance filter for filtration, or the like, and have reached the following disclosure. The present disclosure provides a fiber sheet having excellent quality, a method for manufacturing a fiber sheet, and a cell culture chip.

A fiber sheet according to one aspect of the present disclosure includes:

a first fiber layer including a plurality of first fibers, the plurality of first fibers comprising a thermoplastic polymer and arranged side by side in a first direction;

a second fiber layer including a plurality of second fibers, the plurality of second fibers comprising a thermoplastic polymer and arranged side by side in a second direction intersecting the first direction, and disposed to face the first fiber layer; and

a nanofiber layer including nanofibers, the nanofibers comprising any one of a thermoplastic polymer, a thermosetting polymer, a biodegradable polymer, and a biological polymer, the nanofiber layer being disposed to be in contact with the first fiber layer and the second fiber layer,

in which the nanofiber layer is heat-welded to the first fiber layer and the second fiber layer.

According to this configuration, it is possible to provide a fiber sheet having excellent quality.

The nanofiber layer is disposed between the first fiber layer and the second fiber layer.

Portions at which the plurality of first fibers and the nanofibers are in contact with each other may be heat-welded, and portions at which the plurality of second fibers and the nanofibers are in contact with each other may be heat-welded.

According to this configuration, it is possible to prevent the nanofiber layer from peeling off from the first fiber layer and the second fiber layer.

The second fiber layer may be laminated on the first fiber layer.

The nanofiber layer may be laminated on the second fiber layer.

Portions at which the plurality of first fibers and the plurality of second fibers intersect and are in contact with each other may be heat-welded.

Portions at which the plurality of first fibers and the nanofibers are in contact with each other may be heat-welded.

Portions at which the plurality of second fibers and the nanofibers are in contact with each other may be heat-welded.

According to this configuration, it is possible to prevent the nanofiber layer from peeling off from the first fiber layer and the second fiber layer.

A cross section of each of the plurality of first fibers may have a flat part formed in a flat shape and an arched part formed in an arch shape.

The flat part may be positioned on a side opposite to the second fiber layer.

The arched part may face the second fiber layer.

A cross section of each of the plurality of second fibers may be circular.

According to this configuration, a cell membrane uniform in a plane direction can be cultured.

In the arched part, a contact angle between the plurality of first fibers and a liquid adhering to the plurality of first fibers may be 60° or greater and 150° or smaller.

According to this configuration, it is possible to control spreadability of cells in cell culture.

The thickness of each of the plurality of first fibers may be 1 μm or greater and 50 μm or smaller.

The thickness of each of the plurality of second fibers may be 1 μm or greater and 50 μm or smaller.

According to this configuration, a thin fiber sheet can be provided.

The thermoplastic polymer may be at least any one of polystyrene, polycarbonate, polyethylene terephthalate, polyvinyl chloride, polymethyl methacrylate, and polyamide.

According to this configuration, it is possible to provide a fiber sheet that is thin and has improved in strength.

The thermosetting polymer may be at least one of polyurethane, polyimide, unsaturated polyester resin, epoxy resin, phenol resin, vinyl ester resin, and melamine resin.

According to this configuration, it is possible to provide a fiber sheet having high physical strength and heat resistance.

The biodegradable polymer may be at least any one of polyvinyl alcohol, polyurethane, polylactic acid, polycaprolactone, polyethylene glycol, polylactic acid glycolic acid, ethylene vinyl acetate, and polyethylene oxide.

According to this configuration, it is possible to provide a fiber sheet having high physical strength.

The biological polymer may be at least any one of collagen, gelatin, and cellulose.

According to this configuration, it is possible to provide a fiber sheet having high physical strength.

A method for manufacturing a fiber sheet according to another aspect of the present disclosure includes:

arranging a plurality of first fibers, which comprise a thermoplastic polymer, side by side in a first direction to form a first fiber layer on a surface of a film base material;

forming a nanofiber layer, which includes nanofibers comprising any one of a thermoplastic polymer, a thermosetting polymer, a biodegradable polymer, and a biological polymer, on the first fiber layer;

arranging a plurality of second fibers, which comprise a thermoplastic polymer, side by side in a second direction intersecting the first direction and arranging the plurality of second fibers to face the first fiber layer to form a second fiber layer on the nanofiber layer;

heating the film base material on which the first fiber layer, the nanofiber layer, and the second fiber layer are formed to heat-weld each of portions at which the nanofibers and the plurality of first fibers are in contact with each other and portions at which the nanofibers and the plurality of second fibers are in contact with each other; and

peeling off the film base material from a structure including the first fiber layer, the nanofiber layer, and the second fiber layer, which are heat-welded.

According to this configuration, it is possible to provide a method for manufacturing a fiber sheet having excellent quality.

A method for manufacturing a fiber sheet according to still another aspect of the present disclosure includes:

arranging a plurality of first fibers, which comprise a thermoplastic polymer, side by side in a first direction to form a first fiber layer on a surface of a film base material;

arranging a plurality of second fibers, which comprise a thermoplastic polymer, side by side in a second direction intersecting the first direction and arranging the plurality of second fibers to face the first fiber layer to form a second fiber layer on the first fiber layer;

heating the film base material on which the first fiber layer and the second fiber layer are formed to heat-weld portions at which the plurality of first fibers and the plurality of second fibers intersect and are in contact with each other;

forming a nanofiber layer, which includes nanofibers comprising any one of a thermoplastic polymer, a thermosetting polymer, a biodegradable polymer, and a biological polymer on the first fiber layer and the second fiber layer which are formed on the film base material and heat-welded;

heating the film base material on which the first fiber layer, the second fiber layer, and the nanofiber layer are formed to heat-weld each of portions at which the nanofibers and the plurality of first fibers are in contact with each other and portions at which the nanofibers and the plurality of second fibers are in contact with each other; and

peeling off the film base material from a structure including the first fiber layer, the second fiber layer, and the nanofiber layer, which are heat-welded.

According to this configuration, it is possible to provide a method for manufacturing a fiber sheet having excellent quality.

A cell culture chip according to still another aspect of the present disclosure includes:

the fiber sheet of the above-described aspect.

According to this configuration, it is possible to provide a cell culture chip capable of accurately imitating the function of an organ in a living body.

Hereinafter, exemplary embodiments will be described based on the drawings.

### First Exemplary Embodiment

### Overall Configuration

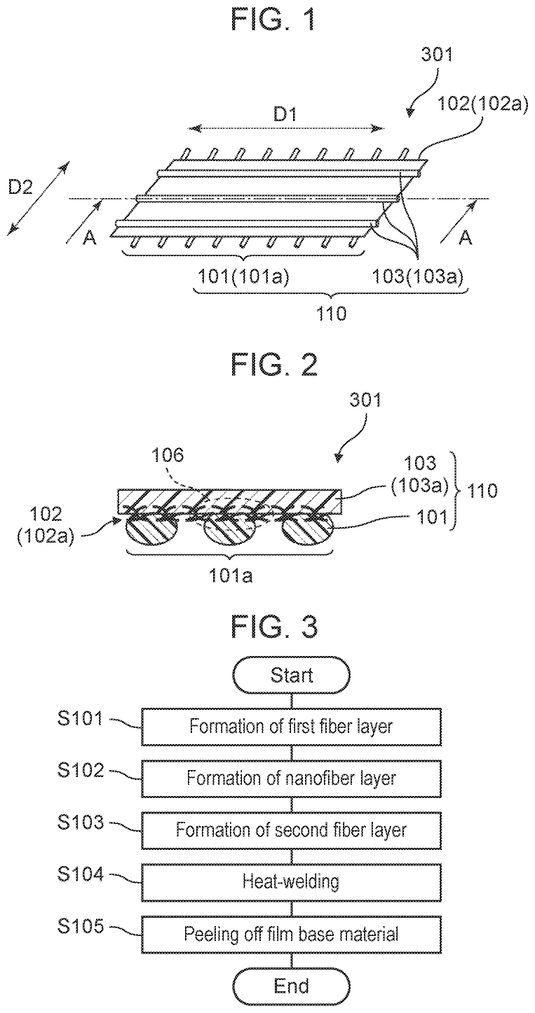

FIG. 1 is a schematic view showing an example of fiber sheet 301 according to a first exemplary embodiment. FIG. 2 is a cross-sectional view taken along a line A-A of fiber sheet 301 of FIG. 1.

Fiber sheet 301 is a sheet used as a scaffolding material in cell culture, a filter for filtration, or the like. As shown in FIG. 1, fiber sheet 301 includes first fiber layer 101a, second fiber layer 103a, and nanofiber layer 102a. In the first exemplary embodiment, first fiber layer 101a and second fiber layer 103a form support base material 110 that supports nanofiber layer 102a.

First fiber layer 101a is formed by arranging a plurality of first fibers 101 formed of a thermoplastic polymer side by side in first direction D1. In first fiber layer 101a, each of the plurality of filamentous first fibers 101 extends along second direction D2 intersecting first direction D1. Each of the plurality of first fibers 101 has, for example, a circular or elliptical cross section. The plurality of first fibers 101 are respectively arranged with intervals therebetween to form first fiber layer 101a. In the present exemplary embodiment, the plurality of first fibers 101 extending in second direction D2 are regularly arranged side by side in first direction D1 at equal intervals to form first fiber layer 101a.

In second fiber layer 103a, a plurality of second fibers 103 formed of a thermoplastic polymer are arranged side by side in second direction D2 intersecting first direction D1 and are arranged to face first fiber layer 101a. In second fiber layer 103a, the plurality of filamentous second fibers 103 extend along first direction D1. Each of the plurality of second fibers 103 have, for example, circular or elliptical cross sections. The plurality of second fibers 103 are respectively arranged with intervals therebetween to form second fiber layer 103a. In the present exemplary embodiment, the plurality of second fibers 103 are regularly arranged side by side in second direction D2 at equal intervals to form second fiber layer 103a.

First fiber layer 101a is an aggregate of first fibers 101, and second fiber layer 103a is an aggregate of second fibers 103. Support base material 110 is a laminate of first fiber layer 101a and second fiber layer 103a.

The thickness of first fiber 101 is preferably 1 μm or greater and 50 μm or smaller. Similarly, the thickness of second fiber 103 is preferably 1 μm or greater and 50 μm or smaller. The thickness of first fiber 101 and second fiber 103 is the length of the widest portion in the cross section of first fiber 101 and second fiber 103. By setting the thickness of first fiber 101 and second fiber 103 within this range, the thickness of fiber sheet 301 can be reduced.

Nanofiber layer 102a contains nanofibers 102 formed of any one of a thermoplastic polymer, a thermosetting polymer, a biodegradable polymer, and a biological polymer. Nanofiber layer 102a is heat-welded to first fiber layer 101a and second fiber layer 103a.

In the present exemplary embodiment, nanofiber layer 102a is disposed between first fiber layer 101a and second fiber layer 103a. Portions at which first fibers 101 and nanofibers 102 are in contact with each other are heat-welded, and portions at which second fibers 103 and nanofibers 102 are in contact with each other are heat-welded.

The thermoplastic polymer is at least any one of polystyrene, polycarbonate, polyethylene terephthalate, polyvinyl chloride, polymethyl methacrylate, and polyamide.

The thermosetting polymer is at least any one of polyurethane, polyimide, unsaturated polyester resin, epoxy resin, phenol resin, vinyl ester resin, and melamine resin.

The biodegradable polymer is at least any one of polyvinyl alcohol, polyurethane, polylactic acid, polycaprolactone, polyethylene glycol, polylactic acid glycolic acid, ethylene vinyl acetate, and polyethylene oxide.

The biological polymer is at least any one of collagen, gelatin, and cellulose.

First fibers 101 and second fibers 103 are arranged to intersect each other. An intersecting angle between each of first fibers 101 and each of second fibers 103 is preferably 30° or greater and 150° or smaller.

Nanofibers 102 and first fibers 101 or second fibers 103 are bonded by heat-welding. In fiber sheet 301, welded portions 106 are formed by bonding portions at which nanofiber layer 102a and first fiber layer 101a or second fiber layer 103a are in contact with each other by heat-welding. Therefore, it is possible to prevent nanofibers 102 from peeling off from support base material 110.

Each of first fibers 101 has a circular or elliptical cross section. Similarly, each of second fibers 103 may have a circular or elliptical cross section. In the present exemplary embodiment, as shown in FIG. 2, first fibers 101 having an elliptical cross section will be described.

### Manufacturing Method

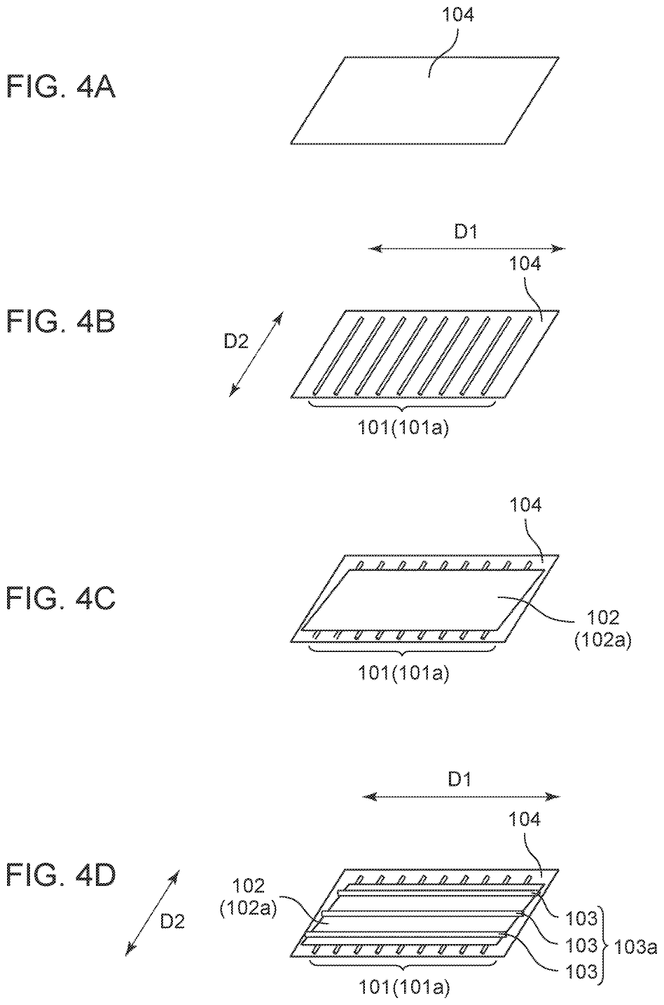

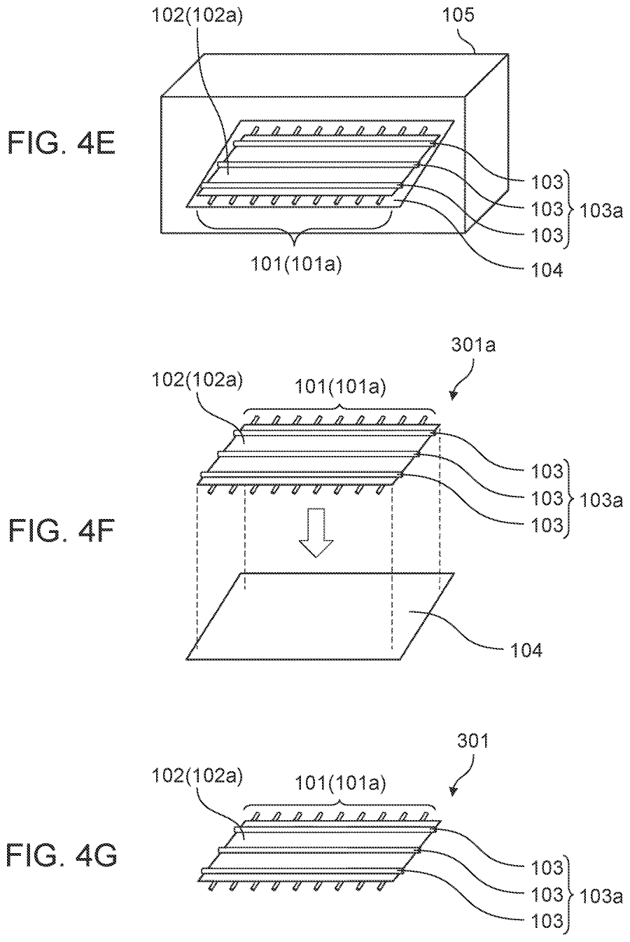

A method for manufacturing fiber sheet 301 will be described with reference to FIGS. 3 to 4G. FIG. 3 is a flowchart showing the method for manufacturing fiber sheet 301 of FIG. 1. FIGS. 4A to 4G are views each showing an example of a manufacturing process of the method for manufacturing fiber sheet 301 of FIG. 3.

First, as shown in FIG. 4A, a film base material 104, which has peelability by being subjected to a peeling treatment such as fluorine processing on the surface, is prepared. Then, as shown in FIG. 4B, a plurality of first fibers 101 formed of a thermoplastic polymer are arranged side by side in first direction D1 to form first fiber layer 101a on the surface of film base material 104 (step S101). First fiber layer 101a can be formed by using a thermoplastic polymer such as polystyrene. First fiber layer 101a is formed by, for example, applying the plurality of first fibers 101 each having a thickness of 2 μm by a dry spinning method. Specifically, first fiber layer 101a is formed by applying the plurality of first fibers 101 extending in second direction D2 to first direction D1 at predetermined intervals. For example, first fibers 101 may be applied at intervals of 10 μm to be arranged in parallel.

Next, as shown in FIG. 4C, nanofibers 102 are applied to first fiber layer 101a to form nanofiber layer 102a (step S102). Nanofiber layer 102a can be formed by applying a polymer to first fiber layer 101a by an electrospinning method using a biodegradable polymer such as polyurethane. Production conditions by the electrospinning method are, for example, a voltage of 20 kV, a distance of 150 mm between a coating nozzle and film base material 104, and a fiber diameter of 500 nm or greater and 900 nm or smaller.

Next, as shown in FIG. 4D, a plurality of second fibers 103 formed of a thermoplastic polymer are arranged side by side in second direction D2 intersecting first direction D1 to form second fiber layer on nanofiber layer 102a (step S103). Second fiber layer 103a can be formed by using a thermoplastic polymer such as polystyrene, as in the case of first fiber layer 101a. Second fiber layer 103a is formed by, for example, applying the plurality of second fibers 103 each having a thickness of 2 μm by a dry spinning method. Specifically, second fiber layer 103a is formed by applying the plurality of second fibers 103 extending in first direction D1 to second direction D2 at predetermined intervals. For example, second fibers 103 may be applied at intervals of 10 μm to be arranged in parallel.

Next, film base material 104 on which first fiber layer 101a, nanofiber layer 102a, and second fiber layer 103a are formed is heated. By heating, portions at which nanofibers 102 and the plurality of first fibers 101 are in contact with each other and portions at which nanofibers 102 and the plurality of second fibers 103 are in contact with each other are heat-welded (step S104). As shown in FIG. 4E, nanofibers 102 are heat-welded to first fibers 101 and second fibers 103 by putting film base material 104 containing first fiber layer 101a, nanofiber layer 102a, and second fiber layer 103a into heating furnace 105 and performing heat treatment. Heating conditions in the heat treatment are, for example, a temperature of 130° C. and a heating time of 20 minutes.

Next, as shown in FIG. 4F, film base material 104 is peeled off from structure 301a including first fiber layer 101a, nanofiber layer 102a, and second fiber layer 103a, which have been heat-welded (step S105).

As shown in FIG. 4G, fiber sheet 301 is completed by the above-described processes.

### Effect

According to the above-described exemplary embodiment, it is possible to provide fiber sheet 301 having excellent quality and the method for manufacturing fiber sheet 301.

In fiber sheet 301, nanofibers 102 are heat-welded to first fibers 101 and second fibers 103. Therefore, nanofiber layer 102a is unlikely to be peeled off, and a fiber sheet having excellent quality can be provided.

In the present exemplary embodiment, nanofiber layer 102a is disposed between first fiber layer 101a and second fiber layer 103a. Therefore, it is possible to prevent nanofiber layer 102a from peeling off from support base material 110 composed of first fiber layer 101a and second fiber layer 103a. Accordingly, when the fiber sheet is used as, for example, a scaffolding material for cell culture, peeled off nanofibers 102 are less likely to become foreign substances, and culture with stable quality is possible.

In the above-described exemplary embodiment, the example in which first fiber layer 101a and second fiber layer 103a are formed by the dry spinning method has been described, but the method for forming first fiber layer 101a and second fiber layer 103a is not limited thereto. For example, it is also possible to use other methods such as a solution spinning method, a dispensing method, or an inkjet method.

In the above-described exemplary embodiment, the example in which the thickness of each of first fibers 101 and each of second fibers 103 is 2 μm has been described, but the thickness is not limited thereto. The thickness of each of first fibers 101 and each of second fibers 103 may be 1 μm or greater and 50 μm or smaller.

In the above-described exemplary embodiment, the example in which the fiber diameter of nanofibers 102 is 500 nm or greater and 900 nm or smaller has been described, but it is sufficient for the fiber diameter of the nanofibers to be within the range of 1 nm or greater and 1000 nm or smaller.

### Modification Example

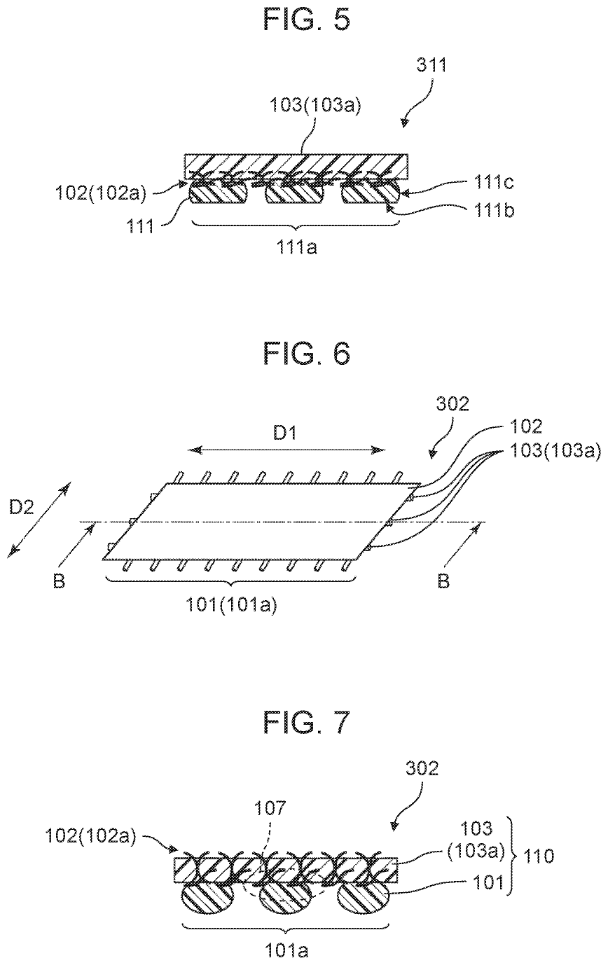

FIG. 5 is a schematic cross-sectional view of fiber sheet 311 according to a modification example of the first exemplary embodiment. As shown in FIG. 5, a cross section of each of a plurality of first fibers 111 may have flat part 111b formed in a flat shape and arched part 111c formed in an arch shape. Flat part 111b is positioned on a side opposite to second fiber layer 103a. Arched part 111c faces second fiber layer 103a. A cross section of each of a plurality of second fibers 103 is circular.

Furthermore, a contact angle may be 60° or greater and 150° or smaller in arched part 111c. The contact angle refers to an angle formed by first fibers 101 and a liquid adhering to first fibers 101. The size of the contact angle can be adjusted by controlling a heating temperature and a heating time in heating furnace 105 (refer to FIG. 4E). For example, when the heat treatment is performed under heating conditions of a temperature of 130° C. and a heating time of 20 minutes, the contact angle of arched part 111c can be set to 120°.

When fiber sheet 311 having such a configuration is used as, for example, a scaffolding material for cell culture, and when cells are seeded on the surface of flat part 111b, the cells spread along flat part 111b of first fiber layer 111a due to the nature of the cells. Therefore, a cell membrane uniform in a plane direction can be obtained.

### Second Exemplary Embodiment

A second exemplary embodiment will be described with reference to FIGS. 6 to 8. In the second exemplary embodiment, the same or equivalent configurations as those in the first exemplary embodiment will be described with the same reference numerals. In the second exemplary embodiment, descriptions overlapping the first exemplary embodiment are omitted.

FIG. 6 is a schematic view showing an example of fiber sheet 302 according to the second exemplary embodiment. FIG. 7 is a cross-sectional view taken along a line B-B of fiber sheet 302 of FIG. 6.

As shown in FIGS. 6 and 7, second fiber layer 103a is laminated on first fiber layer 101a, and nanofiber layer 102a is laminated on second fiber layer 103a, and these are differences from the first exemplary embodiment. In the second exemplary embodiment, portions at which a plurality of first fibers 101 and a plurality of second fibers 103 intersect and are in contact with each other are heat-welded. Portions at which the plurality of first fibers 101 and nanofibers 102 are in contact with each other are heat-welded. Portions at which the plurality of second fibers 103 and nanofibers 102 are in contact with each other are heat-welded.

As shown in FIG. 7, nanofibers 102 and first fibers 101 or second fibers 103 are bonded by heat-welding to form welded portions 107.

As shown in FIG. 7, each of first fibers 101 and each of second fibers 103 are in contact with each other at intersecting portions. Therefore, first fiber layer 101a and second fiber layer 103a are bonded to each other at portions at which the respective fibers thereof intersect, and thereby integral support base material 110 is formed.

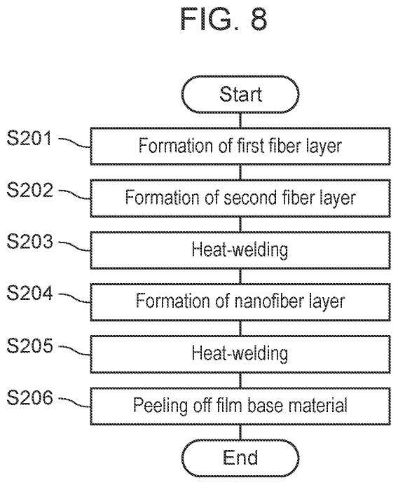

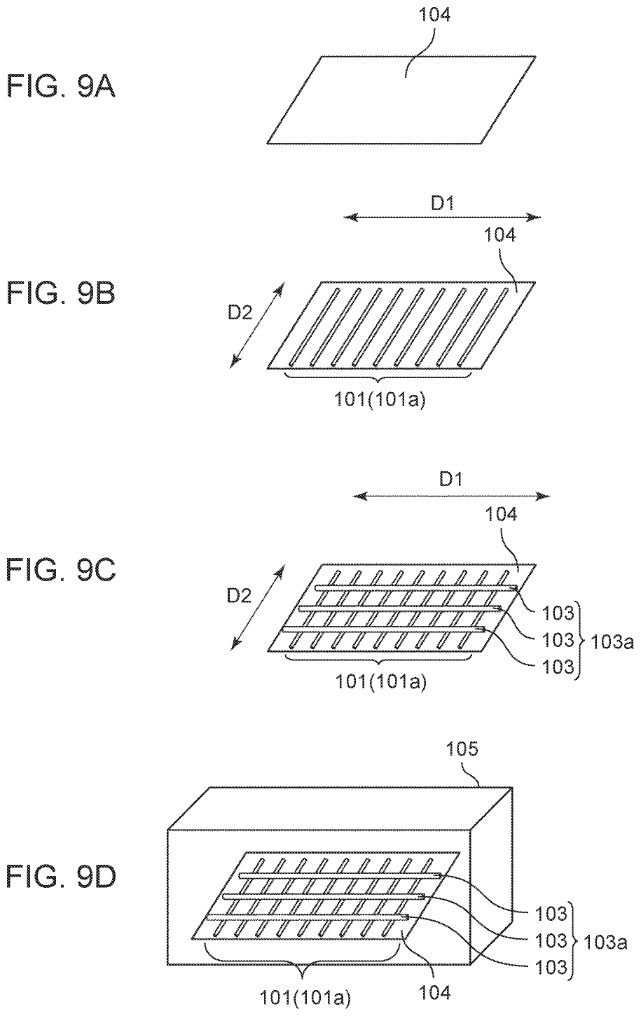

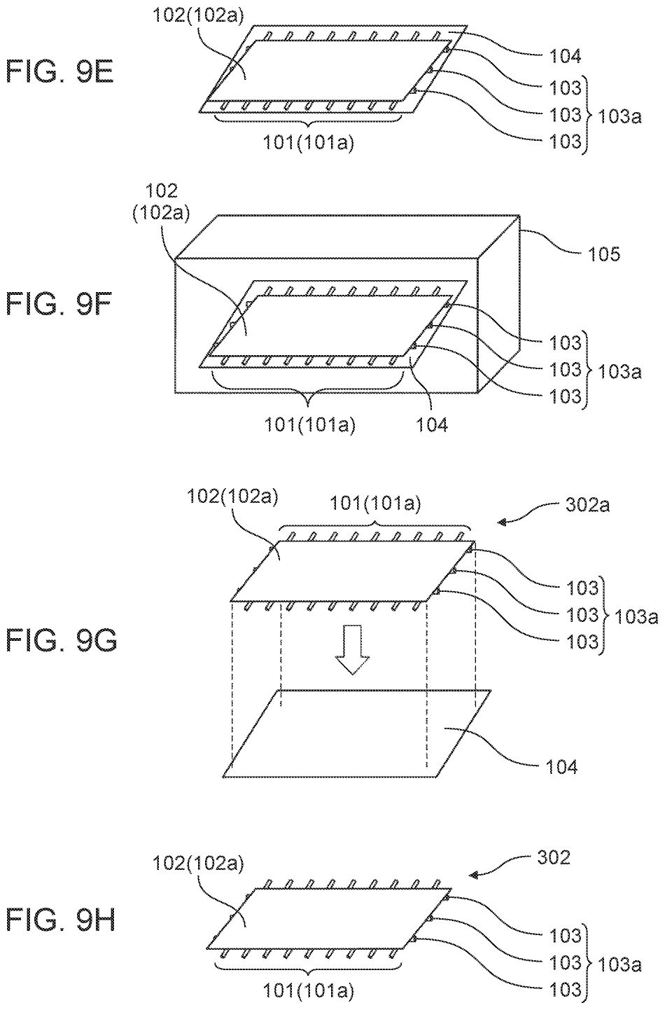

A method for manufacturing fiber sheet 302 will be described with reference to FIGS. 8 to 9H. FIG. 8 is a flowchart showing a method for manufacturing fiber sheet 302 of FIG. 6. FIGS. 9A to 9H are views each showing an example of a manufacturing process of the method for manufacturing fiber sheet 302 of FIG. 8.

As shown in FIG. 8, the present exemplary embodiment differs from the first exemplary embodiment in that second fiber layer 103a is formed instead of nanofiber layer 102a after first fiber layer 101a is formed (step S201), and nanofiber layer 102a is formed after heat-welding is performed. Since the contents of the process in each step are the same as that in the first exemplary embodiment, detailed descriptions thereof will be omitted.

First, as shown in FIGS. 9A and 9B, a plurality of first fibers 101 formed of a thermoplastic polymer are arranged side by side in first direction D1 to form first fiber layer 101a on the surface of film base material 104 (step S201). Next, as shown in FIG. 9C, a plurality of second fibers 103 are arranged on first fiber layer 101a side by side in second direction D2 intersecting first direction D1, and are arranged to face first fiber layer 101a, and thereby second fiber layer 103a is formed (step S202). The plurality of second fibers 103 are formed of a thermoplastic polymer, as in the case of the first fibers.

Next, as shown in FIG. 9D, film base material 104 on which first fiber layer 101a and second fiber layer 103a are formed is heated to heat-weld portions at which the plurality of first fibers 101 and the plurality of second fibers 103 intersect and in contact with each other (step S203). Heating conditions in the heat treatment are, for example, a temperature of 130° C. and a heating time of 20 minutes.

Next, as shown in FIG. 9E, nanofiber layer 102a containing nanofibers 102 is formed on first fiber layer 101a and second fiber layer 103a which are formed and heat-welded on film base material 104 (step S204). Nanofibers 102 are formed of any one of a thermoplastic polymer, a thermosetting polymer, a biodegradable polymer, and a biological polymer.

Next, as shown in FIG. 9F, film base material 104 on which first fiber layer 101a, second fiber layer 103a, and nanofiber layer 102a are formed is heated. By heating, portions at which nanofibers 102 and the plurality of first fibers 101 are in contact with each other are heat-welded. Similarly, portions at which nanofibers 102 and the plurality of second fibers 103 are in contact with each other are heat-welded (step S205). Similar to step S203, heating conditions in the heat treatment are a temperature of 130° C. and a heating time of 20 minutes.

Next, as shown in FIG. 9G, film base material 104 is peeled off from structure 302a including first fiber layer 101a, second fiber layer 103a, and nanofiber layer 102a, which have been heat-welded (step S206).

As shown in FIG. 911, fiber sheet 302 is completed by the above-described processes.

### Effect

According to the above-described exemplary embodiment, the same effect as that of the first exemplary embodiment can be obtained.

### Third Exemplary Embodiment

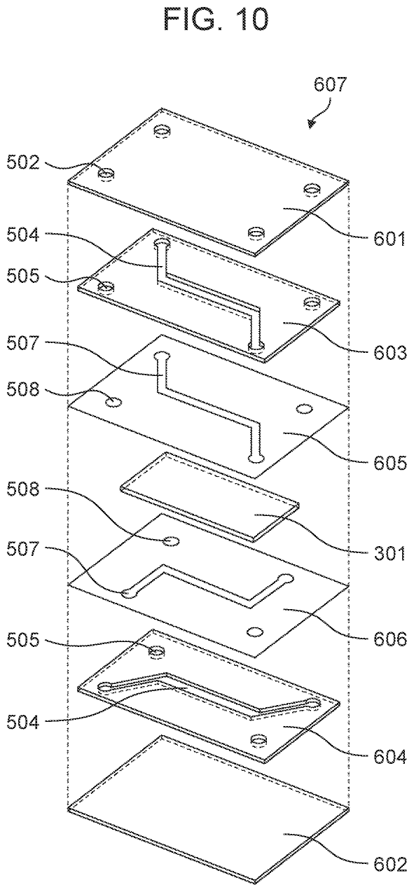

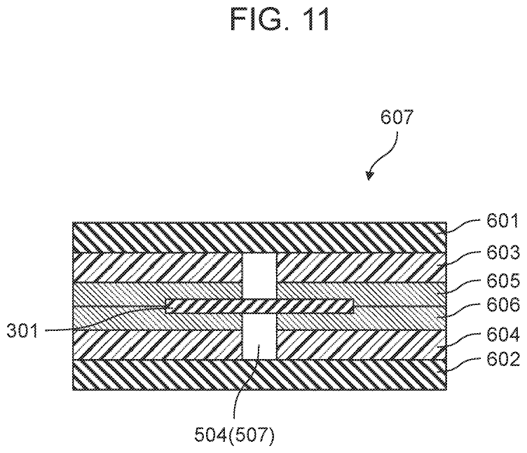

A third exemplary embodiment will be described with reference to FIGS. 10 and 11. In the third exemplary embodiment, cell culture chip 607 in which fiber sheet 301 described in the first exemplary embodiment is used as a scaffolding material will be described. Since fiber sheet 301 is the same as that described in the first exemplary embodiment, the descriptions thereof will be omitted.

FIG. 10 is a schematic decomposition view showing an example of cell culture chip 607 according to the third exemplary embodiment. FIG. 11 is a cross-sectional view of cell culture chip 607 of FIG. 10.

In cell culture chip 607, fiber sheet 301 is used as a scaffolding material. As shown in FIGS. 10 and 11, cell culture chip 607 is configured such that one surface of fiber sheet 301 is adhered to first partition layer 603 via first adhesive layer 605, and the other surface is adhered to second partition layer 604 via second adhesive layer 606. First board 601 is laminated on the outside of first partition layer 603, and second board 602 is laminated on the outside of second partition layer 604.

In each of first partition layer 603 and second partition layer 604, flow path 504 for supplying a liquid medium used for culturing cells is formed. Flow path 504 plays a role for supplying or discharging the medium from the outside of cell culture chip 607. The width of flow path 504 is, for example, 0.3 mm. The width of flow path 504 may be formed within the range of 0.2 to 0.5 mm.

In addition to flow path 504, through holes 505 are formed in each of first partition layer 603 and second partition layer 604. In the present exemplary embodiment, four through holes 505 are formed in each of first partition layer 603 and second partition layer 604. Through hole 505 plays a role as an alignment mark when first partition layer 603 and second partition layer 604 are laminated.

First partition layer 603 and second partition layer 604 can be formed of, for example, a silicone resin.

In each of first adhesive layer 605 and second adhesive layer 606, flow path 507 having a shape corresponding to flow path 504 formed in each of first partition layer 603 and second partition layer 604, and through holes 508 having a shape corresponding to through holes 505 are formed.

Each of first board 601 and second board 602 plays a role as a lid of flow path 504 filled with a liquid medium. Each of first board 601 and second board 602 is made of glass and has a thickness of 0.5 mm. First board 601 and second board 602 can be formed in a thickness within the range of 0.3 to 10 mm. First partition layer 603 and first board 601, and second partition layer 604 and second board 602 are laminated and joined by heat-welding, respectively.

In first board 601, through hole 502 that plays a role as an alignment mark is formed, similarly to first partition layer 603 and second partition layer 604.

As shown in FIG. 11, in the inside of cell culture chip 607, flow path 504 forms a space, and the inside of flow path 504 is filled with a liquid medium. Furthermore, flow path 504 is vertically separated by fiber sheet 301. Therefore, for example, intestinal cells can be cultured on the upper side of fiber sheet 301 (on the side of first partition layer 603), and vascular endothelial cells can be cultured on the lower side of fiber sheet 301 (on the side of second partition layer 604). As described above, according to cell culture chip 607, it is possible to co-culture two types of cultures.

### Effect

According to the above-described exemplary embodiment, it is possible to provide cell culture chip 607 with improved quality.

By using thin fiber sheet 301 as a scaffolding material for cell culture chip 607, an ideal state in which the intestinal cells and the vascular endothelial cells which are disposed above and below the sheet are separated and in contact with each other, can be created. Therefore, it is possible to provide cell culture chip 607 capable of more accurately imitating the function of an organ in a living body.

The present disclosure includes an appropriate combination of any exemplary embodiment among the various exemplary embodiments described above, and the effects of each of the exemplary embodiments can still be exhibited.

According to the fiber sheet, the method for manufacturing a fiber sheet, and the cell culture chip according to the present disclosure, it becomes possible to manufacture and provide a thin fiber sheet having nanofibers and having excellent quality.

What is claimed is:

1. A fiber sheet comprising:
a first fiber layer including a plurality of first fibers, the plurality of first fibers comprising a thermoplastic polymer and arranged side by side in a first direction;
a second fiber layer including a plurality of second fibers, the plurality of second fibers comprising a thermoplastic polymer and arranged side by side in a second direction intersecting the first direction, and disposed to face the first fiber layer; and
a nanofiber layer including nanofibers, the nanofibers comprising any one of a thermoplastic polymer, a thermosetting polymer, a biodegradable polymer, and a biological polymer, the nanofiber layer being disposed to be in contact with the first fiber layer and the second fiber layer,
wherein the nanofiber layer is heat-welded to the first fiber layer and the second fiber layer.

2. The fiber sheet of claim 1,
wherein the nanofiber layer is disposed between the first fiber layer and the second fiber layer, and
portions at which the plurality of first fibers and the nanofibers are in contact with each other are heat-welded, and portions at which the plurality of second fibers and the nanofibers are in contact with each other are heat-welded.

3. The fiber sheet of claim 1,
wherein the second fiber layer is laminated on the first fiber layer,
the nanofiber layer is laminated on the second fiber layer,
portions at which the plurality of first fibers and the plurality of second fibers intersect and are in contact with each other are heat-welded,
portions at which the plurality of first fibers and the nanofibers are in contact with each other are heat-welded, and
portions at which the plurality of second fibers and the nanofibers are in contact with each other are heat-welded.

4. The fiber sheet of claim 1,
wherein a cross section of each of the plurality of first fibers has a flat part formed in a flat shape and an arched part formed in an arch shape,
the flat part is positioned on a side opposite to the second fiber layer,
the arched part faces the second fiber layer, and
a cross section of each of the plurality of second fibers is circular.

5. The fiber sheet of claim 4,
wherein, in the arched part, a contact angle between the plurality of first fibers and a liquid adhering to the plurality of first fibers is 60° or greater and 150° or smaller.

6. The fiber sheet of claim 1,
wherein a thickness of each of the plurality of first fibers is 1 μm or greater and 50 μm or smaller, and
a thickness of each of the plurality of second fibers is 1 μm or greater and 50 μm or smaller.

7. The fiber sheet of claim 1,
wherein the thermoplastic polymer is at least any one of polystyrene, polycarbonate, polyethylene terephthalate, polyvinyl chloride, polymethyl methacrylate, and polyamide.

8. The fiber sheet of claim 1,
wherein the thermosetting polymer is at least one of polyurethane, polyimide, unsaturated polyester resin, epoxy resin, phenol resin, vinyl ester resin, and melamine resin.

9. The fiber sheet of claim 1,
wherein the biodegradable polymer is at least any one of polyvinyl alcohol, polyurethane, polylactic acid, polycaprolactone, polyethylene glycol, polylactic acid glycolic acid, ethylene vinyl acetate, and polyethylene oxide.

10. The fiber sheet of claim 1,
wherein the biological polymer is at least any one of collagen, gelatin, and cellulose.

11. A method for manufacturing a fiber sheet, the method comprising:
arranging a plurality of first fibers, which comprise a thermoplastic polymer, side by side in a first direction to form a first fiber layer on a surface of a film base material;
forming a nanofiber layer, which includes nanofibers comprising any one of a thermoplastic polymer, a thermosetting polymer, a biodegradable polymer, and a biological polymer, on the first fiber layer;
arranging a plurality of second fibers, which comprise a thermoplastic polymer, side by side in a second direction intersecting the first direction and arranging the plurality of second fibers to face the first fiber layer to form a second fiber layer on the nanofiber layer;
heating the film base material on which the first fiber layer, the nanofiber layer, and the second fiber layer are formed to heat-weld each of portions at which the nanofibers and the plurality of first fibers are in contact with each other and portions at which the nanofibers and the plurality of second fibers are in contact with each other; and
peeling off the film base material from a structure including the first fiber layer, the nanofiber layer, and the second fiber layer, which are heat-welded.

12. A method for manufacturing a fiber sheet, the method comprising:
arranging a plurality of first fibers, which comprise a thermoplastic polymer, side by side in a first direction to form a first fiber layer on a surface of a film base material;
arranging a plurality of second fibers, which comprise a thermoplastic polymer, side by side in a second direction intersecting the first direction and arranging the plurality of second fibers to face the first fiber layer to form a second fiber layer on the first fiber layer;
heating the film base material on which the first fiber layer and the second fiber layer are formed to heat-weld portions at which the plurality of first fibers and the plurality of second fibers intersect and are in contact with each other;
forming a nanofiber layer, which includes nanofibers comprising any one of a thermoplastic polymer, a thermosetting polymer, a biodegradable polymer, and a biological polymer on the first fiber layer and the second fiber layer which are formed on the film base material and heat-welded;
heating the film base material on which the first fiber layer, the second fiber layer, and the nanofiber layer are formed to heat-weld each of portions at which the nanofibers and the plurality of first fibers are in contact with each other and portions at which the nanofibers and the plurality of second fibers are in contact with each other; and
peeling off the film base material from a structure including the first fiber layer, the second fiber layer, and the nanofiber layer, which are heat-welded.

13. A cell culture chip comprising:
the fiber sheet of claim 1.
